(19) United States
(12) Patent Application Publication
(10) Pub. No.: US 2013/0096453 A1
(43) Pub. Date: Apr. 18, 2013

(54) BRAIN-COMPUTER INTERFACE DEVICES AND METHODS FOR PRECISE CONTROL

(71) Applicants: Chun Kee CHUNG, Seoul (KR); June Sic KIM, Seoul (KR); Hong Gi YEOM, Seoul (KR)

(72) Inventors: Chun Kee CHUNG, Seoul (KR); June Sic KIM, Seoul (KR); Hong Gi YEOM, Seoul (KR)

(73) Assignee: SEOUL NATIONAL UNIVERSITY R&DB FOUNDATION, Seoul (KR)

(21) Appl. No.: 13/365,318

(22) Filed: Feb. 3, 2012

(30) Foreign Application Priority Data
Oct. 12, 2011 (KR) 10-2011-0104176

(51) Int. Cl. A61B 5/0476

(52) U.S. Cl. 600/544

(57) ABSTRACT

A brain-computer interface device and method for controlling the motion of an object is provided. The brain-computer interface device includes a brain wave information processing unit, which receives converted brain wave information including object motion information, extracts object control information including the object motion information from the converted brain wave information, and transmits the extracted object control information to a hybrid control unit, and a hybrid control unit which receives target information including target location information of a target and outputs final object control information obtained by correcting the object control information including the object motion information based on the target information.

### CROSS-REFERENCE TO RELATED PATENT APPLICATION

This application claims the benefit of Korean Patent Application No. 10-2011-0104176, filed on Oct. 12, 2011, in the Korean Intellectual Property Office, the disclosure of which is incorporated herein in its entirety by reference.

### BACKGROUND OF THE INVENTION

1. Field of the Invention

The present invention relates to brain-computer interface (BCI) devices and methods for precise control of an object to be controlled.

2. Description of the Related Art

Brain-computer interface technology (hereinafter referred to as BCI technology) is a technology that controls a computer or machine by a subject's thought alone. The reason that research institutions have recently recognized the importance and impact of the BCI technology and increased investment therein is that even a paralyzed patient, who cannot move, can express his or her intention, pick up and move an object, or control a transport means, and thus the BCI technology is very useful and necessary. Moreover, the BCI technology is very useful to the public and can be used as an ideal user interface (UI) technology. Thus, the BCI technology can be utilized to control all types of electronic devices such as changing the channel on a television, setting the temperature of an air conditioner, adjusting the volume of music, etc. Furthermore, the BCI technology can be applied to the field of entertainment such as games, the field of military applications, or the elderly who are unable to move, and the social and economic impacts of this technology are very significant.

The BCI technology may be implemented by various methods. A method of using slow cortical potentials, which is used at the initial stage of the research of the BCI technology, utilizes a phenomenon in which the potential of brain waves has a positive or negative value slowly in a one-dimensional operation, such as the distinction between top and bottom, since the potential of brain waves becomes negative due to attention or concentration and otherwise becomes positive. The method of using slow cortical potentials was an innovative method capable of controlling a computer by thought alone at that time. However, the method is not currently used since the response is slow and a high-level of distinction cannot be achieved.

As another method for implementing the BCI technology, a method of using sensorimotor rhythms is one of the most actively pursued research areas. The BCI technology using sensorimotor rhythms is related to the increase and decrease in mu waves (8 to 12 Hz) or beta waves (3 to 30 Hz) according to the activation of the primary sensorimotor cortex and has been widely used to distinguish between left and right.

With the method using the increase and decrease in sensorimotor rhythm, a research group of Berlin, Germany, has succeeded in controlling a mouse cursor with a success rate of 70 to 80% (Benjamin Blankertz et al., 2008).

However, the above-described methods for implementing the BCI technology can only select from a predetermined set of options to the extent of distinguishing between left and right or between top, bottom, left, and right. Moreover, the test is performed within a limited test environment, and thus a BCI technology that provides a more stable and higher recognition rate is required for use in real life.

According to a paper published by BCI group in the UK in Journal of Neural Engineering in 2009, a typing technique with a success rate of 80% or higher through a BCI technology using P300 was shown (M. Salvaris et al, 2009). The P300-based BCI technology uses a positive peak occurring 300 ms after the onset of a stimulus in the parietal lobe, in which the P300 is clearly elicited from a stimulus selected by a subject after various stimuli are sequentially presented to the subject.

Moreover, there is a method known as steady-state visually evoked potential (SSVEP), which has recently attracted much attention. This method utilizes a phenomenon in which the intensity of a frequency increases in the occipital lobe depending on the corresponding frequency of a visual stimulus. According to this method, the classification of signals is relatively easy, and it is possible to select any one of several stimuli at the same time. According to a paper published by the RIKEN laboratory in Japan in Neuroscience Letters in 2010, a method for controlling a mouse cursor by selecting any one of eight directions using the SSVEP was shown (Hovagim Bakardjian et al., 2010).

As such, the BCI technology using the P300 or SSVEP can provide various options, but cannot do anything other than select only one of several predetermined options. Moreover, since the BCI technology requires the visual stimuli, it is impossible to use the BCI technology in daily life, not on the computer.

Moreover, with the typical BCI technologies using brain waves alone, it is very difficult to accurately decode the intension of the subject from the brain waves, and thus the accuracy decreases when an object is controlled using the corresponding brain waves.

### SUMMARY OF THE INVENTION

An object of the present invention is to provide a brain-computer interface device and method which can control an object using brain waves.

Another object of the present invention is to provide a brain-computer interface device and method which can increase the accuracy of control using information of an object when the object is controlled using brain waves.

Still another object of the present invention is to provide a brain-computer interface device and method which can increase the accuracy of control using image recognition of an object when the object is controlled using brain waves.

Yet another object of the present invention is to provide a brain-computer interface device and method which can increase the accuracy of determination of an object using image recognition when the object is controlled using brain waves.

In order to achieve the above-described objects of the present invention, there is provided a brain-computer interface device comprising: a brain wave information processing unit which receives converted brain wave information including object motion information, extracts object control information including the object motion information from the converted brain wave information, and transmits the extracted object control information to a hybrid control unit; and a hybrid control unit which receives target information including target location information of a target and outputs final object control information obtained by correcting the object control information including the object motion information based on the target information.

In the brain-computer interface device, the object may be any one of an artificial arm, a mouse cursor, a control means of an application program displayed on a display, a control means of an audio or, video reproducing device, a wheelchair, and a vehicle.

The brain-computer interface device may further comprise a brain wave signal conversion unit which receives brain wave signals from human, converts the received brain wave signals into converted brain wave information including object motion information, and transmits the converted brain wave information to the brain wave information processing unit.

The brain-computer interface device may further comprise a brain wave signal preprocessing unit which receives the brain wave signals, removes noise signals from the brain wave signals, and transmits the resulting signals to the brain wave signal conversion unit.

The brain-computer interface device may further comprise a target determination unit which receives target information including target location information on at least one target candidate, determines a target, and transmits the determined target information to the hybrid control unit.

The brain-computer interface device may further comprise an image recognition unit which receives an image, extracts at least one target candidate from the received image, sets target information including target location information of the target candidates, and transmits the target information to the target determination unit.

In the brain-computer interface device, the received image may be a stereo image taken by a stereo camera and the target location information may be three-dimensional location information.

In order to achieve the above-described objects of the present invention, there is provided a brain-computer interface method comprising: receiving converted brain wave information including object motion information; extracting object control information including object motion information from the converted brain wave information; receiving target information including target location information on a target; and outputting final object control information obtained by correcting the object control information including the object motion information based on the target information.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram showing a brain-computer interface device in accordance with an exemplary embodiment of the present invention;

FIG. 2 is a schematic diagram showing a control means of an application program by which a target is displayed on a display in a brain-computer interface device in accordance with an exemplary embodiment of the present invention;

FIG. 3 is a schematic diagram showing a brain-computer interface device in accordance with another exemplary embodiment of the present invention;

FIG. 4 is a schematic diagram showing a brain-computer interface device in accordance with still another n exemplary embodiment of the present invention;

FIG. 5 is a block diagram showing a brain-computer interface device in accordance with yet another exemplary embodiment of the present invention;

FIG. 6 is a diagram showing a process of identifying target information by image recognition of received images in accordance with an exemplary embodiment of the present invention;

FIGS. 7 to 9 are flowcharts showing brain-computer interface methods in accordance with exemplary embodiments of the present invention;

FIG. 10 is a diagram showing a process of identifying target information by image recognition of received images in accordance with an exemplary embodiment of the present invention;

FIG. 11 is a diagram showing a process of identifying depth information of objects by image recognition of received stereo images in accordance with an exemplary embodiment of the present invention; and

FIG. 12 is a graph showing object motion information, object location information, and corrected object motion information in accordance with an exemplary embodiment of the present invention.

### DETAILED DESCRIPTION OF THE INVENTION

Hereinafter, reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

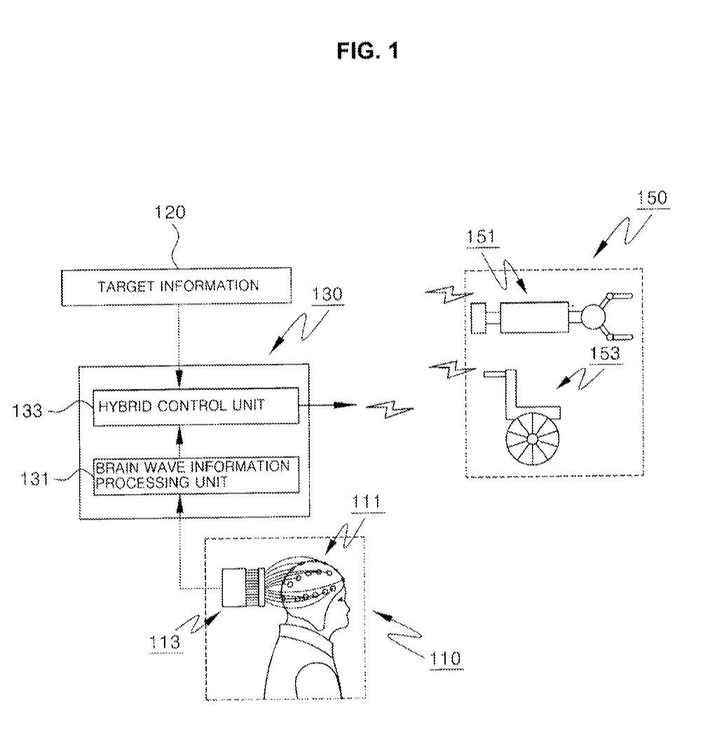

FIG. 1 is a schematic diagram showing a brain-computer interface device 130 in accordance with an exemplary embodiment of the present invention, the brain-computer interface device 130 controlling an object using converted brain wave information of a subject and target information. The functional blocks shown in FIG. 1 and described below are merely possible embodiments. Other functional blocks may be used in other embodiments without departing from the spirit and scope of the invention as defined in the detailed description. Moreover, although at least one functional block of the brain-computer interface device 130 is expressed as individual blocks, the at least one of the functional blocks may be a combination of various hardware and software components that execute the same function.

In the present invention, the brain waves represent electromagnetic signals changed by the activation and state of the brain of the subject. According to exemplary embodiments, the brain waves may include the following brain wave signals according to the measurement method.

Electroencephalogram (EEG) signals are measured from potential fluctuations occurring in the brain of human or animal or brain currents generated thereby by recording from electrodes placed on the scalp.

Magnetoencephalogram (MEG) signals are recorded from biomagnetic fields produced by electrical activity in the brain cells via SQUID sensors.

Electrocorticogram (ECoG) signals are measured from potential fluctuations occurring in the brain or brain currents generated by recording from electrodes placed on the surface of the cerebral cortex.

Near infrared spectroscopy (NIRS) signals are measured by shining light in the near infrared part of the spectrum through the skull and detecting how much the remerging light is attenuated.

In the present invention, it should be understood that while the brain wave signals such as EEG, MEG, and ECoG signals are exemplified in the specification, the brain wave signals are not limited to specific types of brain wave signals, but include all signals generated from the brain of human and measured from the scalp.

Referring to FIG. 1, a brain wave information processing unit 131 of the brain-computer interface device 130 may receive converted brain wave information including object motion information.

An object represents a thing that a subject, from whom brain wave signals or converted brain wave information is measured, wants to control using the brain wave signals or converted brain wave information.

In the present invention, the object is not particularly limited, but may be any one of an artificial arm 151 or 351, a mouse cursor of a display, a control means 235 of an application program displayed on a display, a wheelchair 153, and a vehicle.

The converted brain wave information represents information obtained by extracting information, which includes motion information of an object (i.e., object motion information) that the subject wants to control, from the brain wave signals of the subject such as EEG, MEG, and ECoG signals and by including the object motion information of the extracted object. That is, the converted brain wave signal information means the information converted from the brain wave signals, such as EEG, MEG, and ECoG signals, into the form of a signal that can be recognized by a control device such as a computer, and the converted brain wave information includes the object motion information.

The EEG signals may be measured by electrodes 111, 311 and 411 attached to the scalp of the subject and may be captured by the conventional methods of measuring the MEG and ECoG signals. That is, the brain wave signals may be measured by any one of brain activity measurement devices such as EEG, MEG, ECoG, NIRS, etc.

The measured brain wave signals may be converted into the converted brain wave information including the object motion information by an interface device 113 such as a computer and input to the brain wave information processing unit 131.

For example, the interface device 113 may measure the EEG signals from the subject, perform preprocessing such as digital conversion, noise removal, etc. on the EEG signals, extract predetermined feature vectors, extract the object motion information that the subject wants to control by applying an artificial intelligence method such as regression, artificial neural network, etc. using the feature vectors, and convert the EEG signals into the converted brain wave information including the object motion information.

The object motion information represents all information indicating the motion of the object. For example, when the object is an artificial arm 151, 351 or 451, the object motion information may include all motion information such as vector information from the current location of the artificial arm to a destination location, movement speed information of the artificial arm, etc.

As an example, the object motion information may be object motion information such as “raising up” the object such as the artificial arm or “moving forward” the object such as the wheelchair. In the former case, the object motion information may be obtained using a predetermined code such as “UP” and, in the latter case, the object motion information may be obtained using a predetermined code such as “FORWARD”. The information including the object motion information may be configured as the converted brain wave information.

As another example, the object motion information may include the vector information from the current location of the artificial arm to a destination location. In the case where the object is an artificial arm and the motion vector of the artificial arm is to move from the current location of the artificial arm to 30 cm in the X-axis direction, 60 cm in the Y-axis direction, and 40 cm in the Y-axis direction, the object motion information may be configured as “X:30-Y:60-Z:40”.

Moreover, if the objection motion information includes, for example, the movement speed information of the artificial arm (e.g., the speed is 80 cm/min), the object motion information may be configured with the motion vector of the object as “X:30-Y:60-Z:40, V:80”.

The speed information may be expressed as absolute velocity information (e.g., 80 cm/min) or may be configured as “FAST”, “SLOW”, and “MEDIUM” by classifying the speed information into units of predetermined speeds.

For example, in the case where the object is a wheelchair 153 and it is determined that the subject's intention is to move forward the wheelchair at a high speed, the object motion information may be configured in units of predetermined speeds or configured as the converted brain wave information including the object motion information such as “FORWARD FAST”.

Moreover, if a plurality of objects are connected to the control device, and if there are a plurality of objects that the subject can control at the same time, the converted brain wave information may include the object motion information and information about which object the subject wants to control.

An example of the case where a plurality of objects are connected to the control device and there are a plurality of objects that the subject can control at the same time will be described below.

In the case where the object is an artificial arm 151 and the extracted object motion information is “UP”, if the code of the object such as the artificial arm is predetermined as “ARM” in the control device, the converted brain wave information may include the object code such as “ARM UP” and the object motion information.

In the case where the object is a wheelchair 153 and the extracted object motion information is “FORWARD”, if the code of the object such as the wheelchair is predetermined as “WHEELCHAIR” in the control device, the converted brain wave information may include the object code such a′s “WHEELCHAIR FORWARD” and the object motion information.

The converted brain wave information represents the information including the motion information of the object (i.e., object motion information), and thus the converted brain wave information may include information such as ID, sex, age, etc. of the subject.

Therefore, it will be understood that the brain-computer interface device of the present invention may receive a plurality of brain wave information converted from brain wave signals detected from a plurality of subjects and control the plurality of objects.

The brain wave information processing unit 131 extracts object control information including the object motion information, such as “ARM UP” or “WHEELCHAIR FORWARD”, from the converted brain wave signals and transmits the extracted object control information to a hybrid control unit 133.

The object control information represents information relating only to the control of the object extracted from the converted brain wave information for the control of the object.

For example, if a plurality of objects are connected to the control device, if there are a plurality of objects that the subject can control at the same time, and if the converted brain wave information, which includes the ID of the subject (e.g., “A123”), the sex of the subject (e.g., “MALE”), the object code (e.g., “ARM”), and the object motion information (e.g., “UP”), is “A123-MALE-ARM-UP”, the object control information may be “ARM-UP” by extracting the object code and the object motion information other than the ID and sex of the subject from the converted brain wave information.

The hybrid control unit 133 corrects the object control information transmitted from the brain wave information processing unit 131 based on input target information of a target. The input target information may be target information of at least one target. The target information may include target location information and target recognition information.

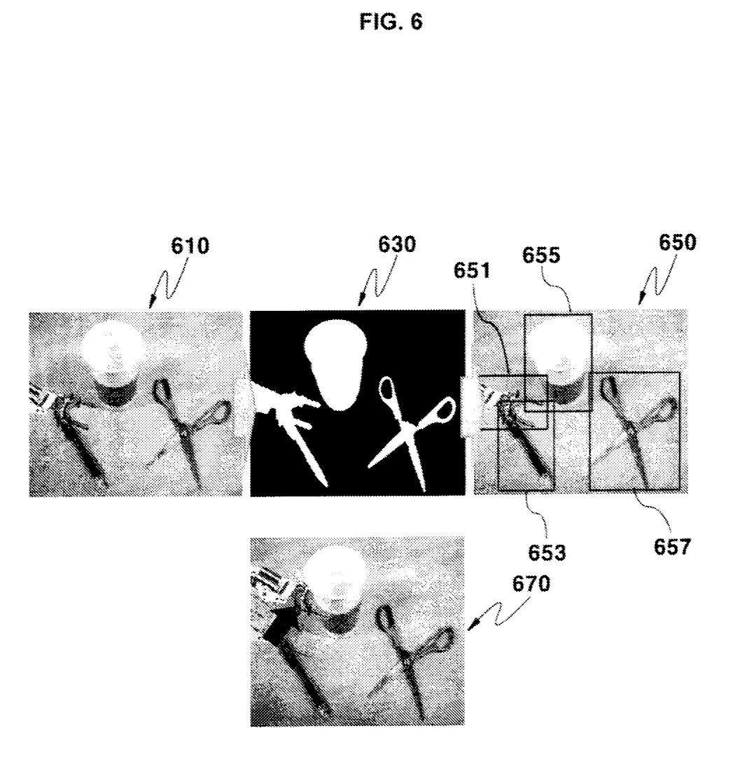

The target represents a target of the controlled object's motion. Referring to FIG. 6, if the final control target of the artificial arm is to take a cup 655 (A), the corresponding cup 655 may be the target. Moreover, if the movement target of the wheelchair is point B, the corresponding point B may be the target.

The target location information represents three-dimensional location information of the target and may be determined as the location of the target identified by image recognition, near field communication, etc. The target recognition information represents information for distinguishing between a unique target candidate and a target.

For example, if the relative three-dimensional location of target A from the artificial arm as the control object is 30 cm in the X-axis direction, 50 cm in the Y-axis direction, and 40 cm in the Y-axis direction, the target information of target-A may be configured as “TARGET-A, X:30-Y:50-Z:40” including the target recognition information and the target location information.

The hybrid control unit 133 corrects the object control information extracted from the converted brain wave information of the subject based on the input target information and outputs final object control information.

The final object control information represents the information obtained by correcting the object control information based on the target information.

For example, in the case where the object motion information of the extracted object control information is “ARM-UP” and the target location information of target A is “X:30-Y:50-Z:40”, the final object control information may be configured as “ARM-UP, TARGET-A, X:30-Y:50-Z:40” including the object control information such as “ARM-UP” and the object information with the target recognition information such as “TARGET-A, X:30-Y:50-Z:40”.

Moreover, the object motion information of the object control information input in the control unit may include motion vector information from the current location to a destination location. For example, in the case where the object is an artificial arm and the motion vector of the artificial arm is to move from the current location of the artificial arm to 30 cm in the X-axis direction, 60 cm in the Y-axis direction, and 40 cm in the Y-axis direction, the object motion information may be configured as “ARM, X:30-Y:60-Z:40”.

In this case, if the target location information of target A is “X:30-Y:50-Z:40”, the object control information “ARM, X:30-Y:60-Z:40” may be corrected to the final object control information “ARM, TARGET-A, X:30-Y:50-Z:40” based on the target information “TARGET-A, X:30-Y:50-Z:40”. Otherwise, the object control information may be corrected to the final object control information “ARM, TARGET-A, X:30-Y:55-Z:40” using an intermediate value of the object motion information of the object control information and the target location information.

Moreover, when the target information of a plurality of targets A and B is received, the object control information may be corrected to an intermediate location of targets A and B based on the target information of the plurality of targets A and B or may be corrected to the final object control information based on the target information of target A or B, which is located more adjacent to the motion vector location of the object control information.

For example, if the object control information “ARM, X:30-Y:60-Z:40” is corrected based on the target information of targets A and B such as “TARGET-A, X:30-Y:50-Z:40” and “TARGET-A, X:30-Y:70-Z:60”, the final object control information may be determined as “ARM, X:30-Y:60-Z:50” based on the intermediate location of targets A and B “X:30-Y:60-Z:50”. Otherwise, if there are a plurality of targets, the object control information may be corrected using a geometric average or arithmetic average, not a simple average of the target locations.

Furthermore, the object control information may be corrected to the final object control information based on the target information using a Kalman filter, extended Kalman filter as the nonlinear version of the Kalman filter, unscented Kalman filter, particle filter, Bayesian filter, etc. which are algorithms for producing closer values to the true values from measurements observed.

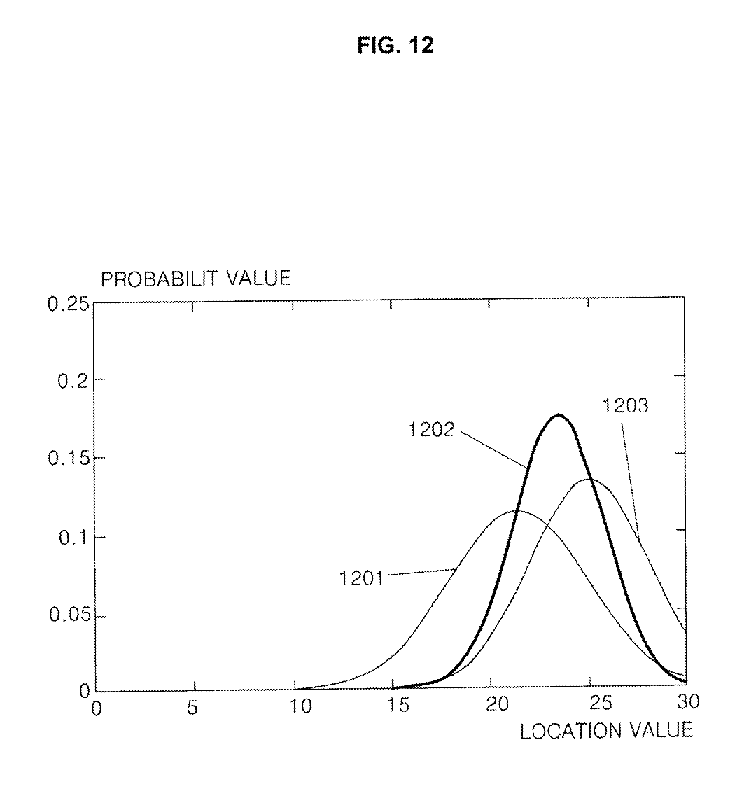

As shown in FIG. 12, the target location information of the target information or the object motion information may be expressed as the distribution of probability values, not as simple numerical values. For example, the X-axis motion information of the object motion information may be expressed as the distribution 1201 of probability values according to the X-axis location variation, and the target location information of the target information may be expressed as the distribution 1203 of probability values according to the X-axis location variation. In this case, the final object control information may be obtained by correcting the object control information based on volume distribution and may also be determined as the distribution 1202 of probability values according to the X-axis location variation. The control information on the Y-axis and Z-axis of the final object control information may be determined in the same manner.

The final object control information may be continuously changed and determined based on the movement of the object, the change of the object control information extracted from the converted brain wave information, and the resulting change of the target information of target candidates.

For example, if the object control information “ARM, X:30-Y:60-Z:40” is corrected based on the target information of target A such as “TARGET-A, X:30-Y:50-Z:40”, the final object control information may be determined by correcting the object control information to “ARM, TARGET-A, X:30-Y:50-Z:40”. Therefore, the artificial arm as the object is moved to target A based on the motion vector “X:30-Y:50-Z:40”, and the object control information, which is extracted from the converted brain wave information input during the movement as the brain waves of the subject change, may change. In the case where the changed object control information is “ARM, X:30-Y:20-Z:40” and the input target information is changed to the information on target B, the object control information may be corrected based on the target information of target B, and the final object control information may be changed and determined as “ARM, TARGET-B, X:30-Y:30-Z:40”.

Moreover, when the algorithm for producing closer values to the true values from measurements observed is used, if the object control information “ARM, X:30-Y:60-Z:40” is corrected based on the target information of target A such as “TARGET-A, X:30-Y:50-Z:40”, the final object control information may be determined by correcting the object control information to “ARM, X:30-Y:55-Z:40” according to the use of the algorithm such as the Kalman filter. Therefore, the artificial arm as the object is moved based on the motion vector “X:30-Y:55-Z:40”, and the object control information, which is extracted from the converted brain wave information input during the movement as the brain waves of the subject change, may change again. In the case where changed object control information is “ARM, X:30-Y:20-Z:40” and the input target information is changed to the information on target B, the object control information may be corrected based on the target information of target B “TARGET-B, X:30-Y:40-Z:40” according to the use of the algorithm such as the Kalman filter, and the final object control information may be changed and determined as “ARM; X:30-Y:30-Z:40”.

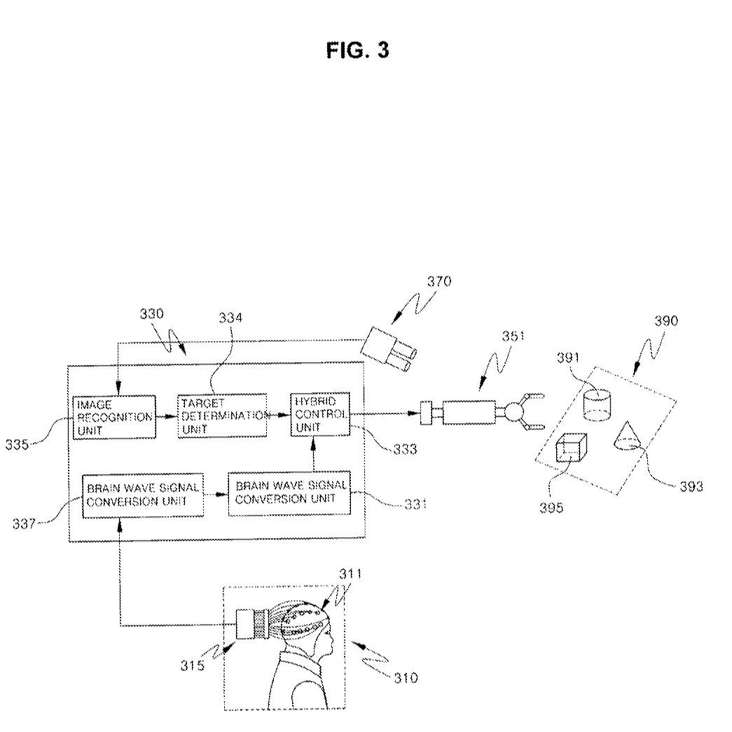

Referring to FIG. 3, the brain-computer interface device may further comprise a brain wave signal conversion unit 337 which receives brain wave signals from human, converts the received brain wave signals into converted brain wave information including object motion information, and transmits the converted brain wave information to the brain wave information processing unit.

The brain wave signal conversion unit 337 may comprise a signal processing unit performing a feature extraction process on the received brain wave or the brain wave signals subjected to preprocessing such as noise removal, etc. and a data classification unit performing a process of determining the object motion information based on the extracted features.

The received brain wave signals or the brain wave signals from which noise signals are removed may be transmitted to the signal processing unit of the brain wave signal conversion unit, and the signal processing unit extracts the features of a signal useful to recognize the subject's intention. The signal processing unit may perform epoching for dividing the brain wave signals into specific regions to be processed, normalization for reducing the difference in brain wave signals between humans and the difference in brain wave signals in a human, and down sampling for preventing overfitting. The epoching is for real-time data processing and may be used in units of several tens of milliseconds to seconds, and the down sampling may be performed at suitable intervals of about 20 ms, but the intervals may vary from several to several tens of ms depending on the subject or conditions. According to circumstances, the signal processing unit may perform a Fourier transform or a signal processing for obtaining an envelope.

The data classification unit identifies the subject's intention reflected in the brain wave signals and determines the type of control for the object. In detail, the data classification unit may determine feature parameters from training data through a data training process and determine appropriate object motion information on new data based on the determined feature parameters. In order to determine the feature parameters from the training data and determine an appropriate output for new data, the data classification unit may use regression methods such as multiple linear regression, support-vector regression, etc., in which classification algorithms such as artificial neural network, support-vector machine, etc. may be employed.

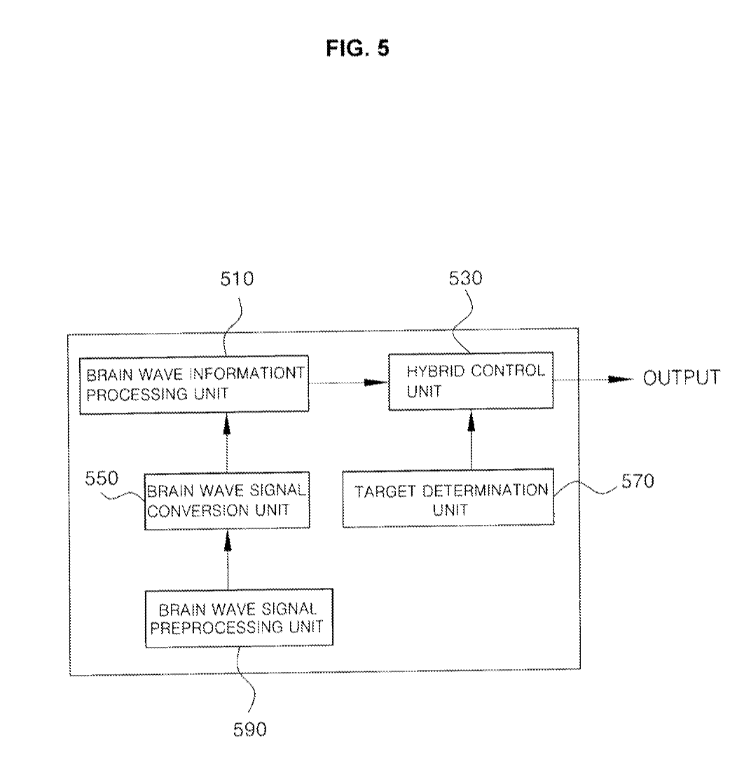

Referring to FIG. 5, the brain-computer interface device may further comprise a brain wave signal preprocessing unit 590. The brain wave signal preprocessing unit may receive brain wave signals, remove noise signals from the brain wave signals, and transmit the resulting signals to the brain wave signal conversion unit.

The brain wave signal preprocessing unit 590 may comprise any one of a low-pass filter, a high-pass filter, a band-pass filter, and a notch filter and may also comprise a device for performing independent component analysis (ICA) or principal component analysis (PCA) to remove noise signals present in the brain wave signals.

The noise signal represents a signal other than the brain wave signals. For example, other biological signals than the brain wave signals such as electromyogram (EMG), electrooculogram (EOG), etc. in addition to the noise signals according typical transmission paths (such as wired and wireless channels) are not of interest and thus may be removed by filtering, for example.

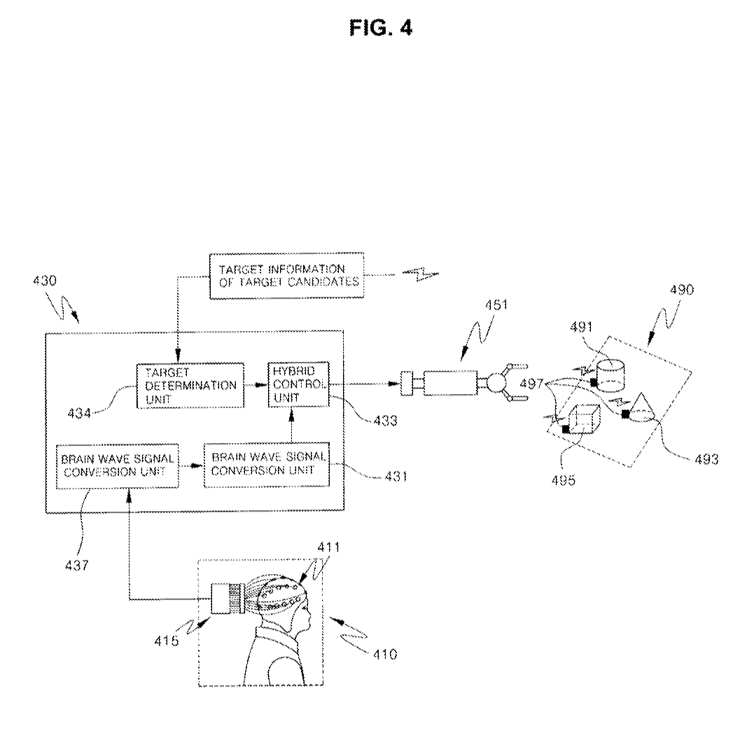

Referring to FIG. 4, the brain-computer interface device may further comprise a target determination unit 434. The target determination unit may receive target information including target location information on at least one target candidate, determine a target, and transmit the determined target information to the hybrid control unit.

The target candidate represents an object that can be determined as a target. The target candidate may be determined by image recognition, Zigbee, ubiquitous sensor network (USN), radio frequency identification (RFID), near field communication (NFC), etc.

The target information may include target location information and target recognition information. The target recognition information represents information for distinguishing between a unique target candidate and a target. For example, if it is identified by the image recognition and near field communication that there are three objects of A, B, and C in the motion direction of the artificial arm (or in a direction that the subject, from whom the brain wave signals are measured, faces), the A, B, and C objects may be recognized as target candidates. In this case, predetermined identifiers of A, B, and C such as “TARGET-A”, “TARGET-B”, and “TARGET-C” may be determined as the target recognition information. Moreover, the location of each of the target candidates A, B, and C identified by the image recognition and near field communication may be determined as the target location information.

Referring back to FIG. 3, if it is identified by automatic image recognition that there are three objects 391, 393, and 395 in an image taken in a direction that the subject, from whom the brain wave signals are measured, faces, the three objects 391 (A), 393 (B), and 395 (C) may be recognized as the target candidates.

Moreover, referring to FIG. 4, in which the near field communication is used, when an RFID electronic tag, NFC tag, Zigbee chip, USN sensor, etc. is attached to each object 490, the location of each object present within a predetermined range around the subject, from whom the brain wave signals are measured, can be identified. Thus, it is possible to recognize the related objects 490 as the target candidates based on the location of the subject, from whom the brain wave signals are measured, and the location and movement direction of the object to be controlled.

The target determination unit may determine a target from at least one target candidate based on the location of the subject, from whom the brain wave signals are measured, and the location and movement direction of the object to be controlled.

As an example, referring to FIG. 4, if the objects 491 (A), 493 (B), and 495 (C) identified by the near field communication are recognized as surrounding objects of the subject, from whom the brain wave signals are measured, the objects A and B may be recognized as the target candidates based on the facing direction of the subject and the direction of the object to be controlled. In this case, the target determination unit may determine the target candidate, which is closest to the current location of the artificial arm 451 as the object, from the target candidates as the final target or may determine the target candidate, which is located in an extending direction of the current movement of the object, as the target candidate.

As another example, referring to FIG. 4, the final target may be determined by referring to the object control information extracted from the converted brain wave information and based on the movement direction and speed. For example, a case where the object is an artificial arm, the target candidates are A, B, and C, and the object control information is “X:10-Y:10-Z:10” will be described. When the movement speed of the object is low, even if all candidates A, B, and C are present within a predetermined range from the movement direction of the object, the closest target candidate C may be recognized as the final target. On the contrary, when the movement speed of the object is high, the farthest target candidate B may be recognized as the final target.

As another example, referring to FIG. 4, when the object control information and the target location information of the target candidates are taken into account, a plurality of target candidates may be determined as the targets. For example, in the case where the object is an artificial arm and the target candidates are A, B, and C, if it is determined that target candidates A and B are closely related to each other based on the object control information, both target candidates A and B may be determined as the targets. In this case, the brain wave signals from the subject and the resulting converted brain wave signals vary over time, and thus one target may be finally determined based on the movement of the object.

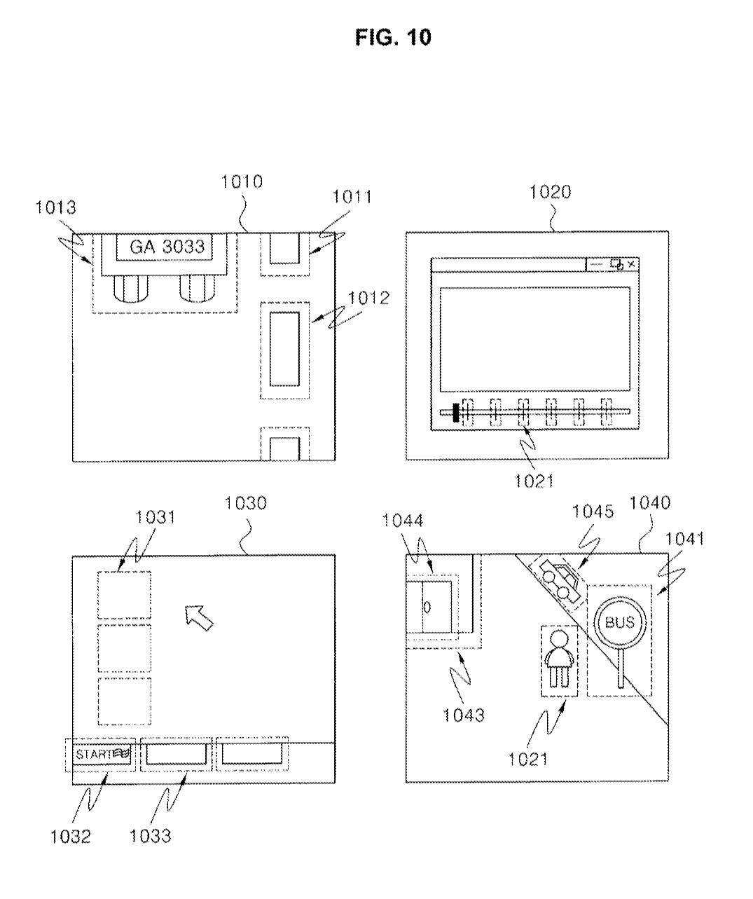

As another example, referring to FIG. 10, the target candidate may be determined based on the conditions of the subject and the object. For example, in case 1010 where the object is a vehicle and there are a plurality of target candidates recognized from a received image, a preceding vehicle 1013 and a centerline mark may not be determined as the target based the fact that that the object is the vehicle.

Otherwise, in case 1040 where the object is a wheelchair and there are a plurality of target candidates recognized from a received image, a vehicle 1045 on a road and a surrounding person 1042 may not be determined as the target based the fact that that the object is the wheelchair.

In a case where the object is a volume or controller of a video program displayed on a display, the target may be determined from a level indicator 1021 related to the corresponding controller based the object.

Moreover, since the brain wave signals from the subject, the resulting converted brain wave signals, and the surrounding conditions may vary continuously, it is natural that the target candidates and the determined target vary.

Although a target candidate suitable for the above description and predetermined criteria may be determined as the target, the target candidate may be determined by applying an artificial intelligence method such as artificial neural network, for example.

Referring to FIG. 3, the brain-computer interface device may further comprise an image recognition unit 335. The image recognition unit receives an image, extracts at least one target candidate from the received image, sets target information including target location information of the target candidates, and transmits the target information to the target determination unit. The target information may include target recognition information.

The image recognition unit may receive an image from an external camera 370 or receive an image through another transmission device.

The received image is a surrounding image of the subject, from whom the brain wave signals are measured, and in particular a surrounding image in the direction of the subject's head or eyes may be suitable.

Referring to FIG. 10, the received image is not limited to images 1010 and 1040 taken by a camera, but may include all images such as captured images 1020 and 1030 on a display.

The image recognition unit 335 may set the target information including the target recognition information and target location information of the target candidates based on information on the location and shape of the objects identified from the received image and may transmit the target recognition information to the target determination unit 334.

The image recognition unit 335 may perform an image processing process through linear spatial filtering techniques such as low-pass filtering, high-pass filtering, etc. or an image preprocessing process through non-linear spatial filtering techniques such as maximum filtering, minimum filtering, etc.

The image recognition unit 335 may obtain the shape of an object present in the image in combination with methods such as thresholding for dividing the received image into two regions based on thresholds, Harris corner detection, difference image or color filtering and may identify the location of the object present in the image by applying an image processing technique of clustering the objects using unsupervised learning such as K-means algorithm.

For example, the target candidates in FIG. 6 may include a pen 653, a cup 655, and a pair of scissors 657 recognized from the received image through the above-described image processing process. Thus, the target information including the target recognition information and target location information of the recognized pen 653, cup 655, and scissors 657 may be set and transmitted to the target determination unit 334.

Moreover, although the image recognition unit may set the target information by recognizing all of the objects in the received image as the target candidates as mentioned above, the image recognition unit may recognize a portion of the objects in the received image as the target candidate based on various conditions such as the direction of the subject's eyes, the direction of the object to be controlled, etc.

It should be noted that the target candidates may be newly recognized according to the change of the conditions. For example, when the brain wave information converted from the brain wave signals of the subject is compared with the converted brain wave information before a predetermined time, if the converted brain wave information is changed to a predetermined value, if the direction of the subject's head or eyes is changed beyond a predetermined range, or if the object information of the object identified by the image recognition and near field communication is changed to a predetermined value, the target candidates may be newly recognized.

Otherwise, if it is determined that the target for the object to be controlled by the subject is changed by comprehensively determining the above exemplified cases, without separately determining the cases, the target candidates may be newly recognized.

In order to determine whether the conditions for identifying the target candidates are changed, a change above a predetermined value may be determined as the change in conditions, and the change in conditions may be determined by applying an artificial intelligence method such as artificial neural network, for example.

As shown in FIG. 10, the image recognition unit may recognize lane marks 1011 and 1012 on a road, a volume of an application program displayed on a display 1020 or a level indicator 1021 around a controller, an icon 1031 around a mouse pointer displayed on a display 1030, and clickable objects 1032 and 1033, which are distinguishable from the background, as the target candidates, as well as the objects shown in FIG. 6.

Moreover, the image recognition unit may recognize the objects present in the received image as the target candidates based on the conditions of the objects. For example, in the case where the object is a vehicle running on a road in FIG. 10, another vehicle 1013 preceding the object and the lane mark 1012 may be recognized as the objects, but they may not be recognized as the target candidates based the fact that the vehicle in front is located too close or that the object is the vehicle based on the conditions in which the vehicle as the object is running.

Similarly, in the case where the object is a wheelchair on a sidewalk in FIG. 10, a bus stop sign 1041, a person 1042 standing on the sidewalk, a vehicle 1045 on a road may be recognized as the sounding objects, but the person 1042 standing on the sidewalk and the vehicle 1045 on the road may not be recognized as the target candidates based the fact that the wheelchair is the object.

Moreover, even in this case, a license plate of another vehicle may not be recognized as the target candidate, although it can be distinguished from the background, as the vehicle 1013 is recognized as the target candidate based on the received image and the conditions of the object.

Referring to FIG. 3, the image recognition unit 335 of the brain-computer interface device may receive a stereo image taken by a stereo camera and set target information including target location information based on three-dimensional location information of objects extracted from the stereo image.

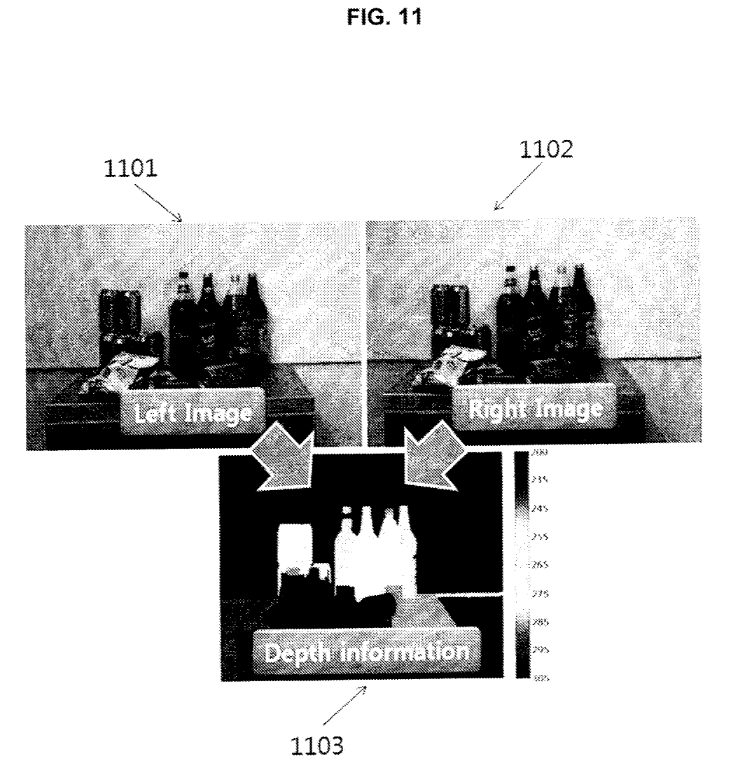

Referring to FIG. 11, the image recognition unit may obtain three-dimensional location information of objects by obtaining depth information 1103 of the objects by image matching, for example, and set target information including target location information based on the three-dimensional location information.

The object of the brain-computer interface device may be any one of an artificial arm, a mouse cursor, a control means of an application program displayed on a display, a control means of an audio device, a wheelchair, and a vehicle.

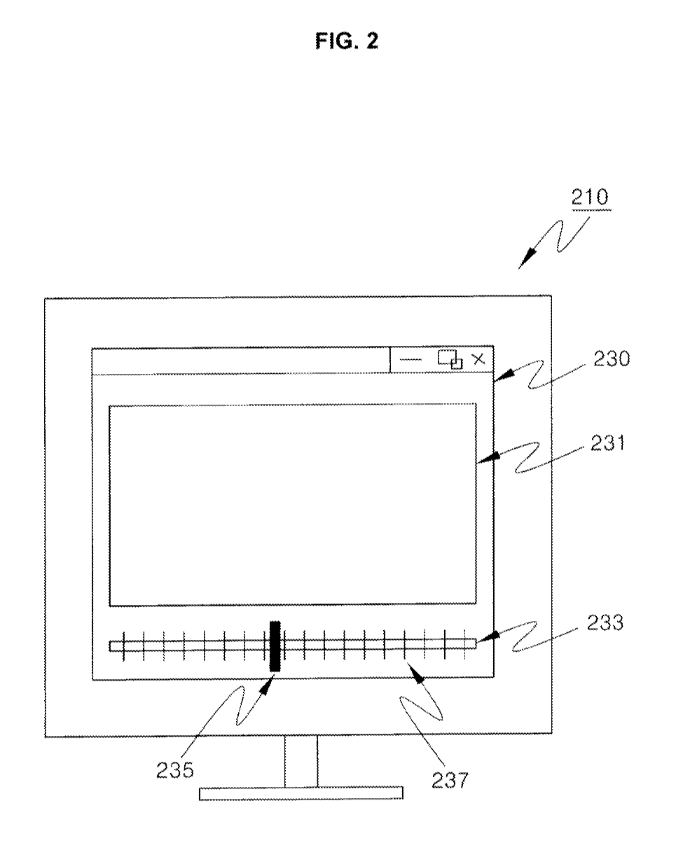

Referring to FIG. 2, in the case where an application program 230 displayed on a display 210 is a video reproducing program or music reproducing program, the object may be a volume control means and a reproduction control means 235 in each program.

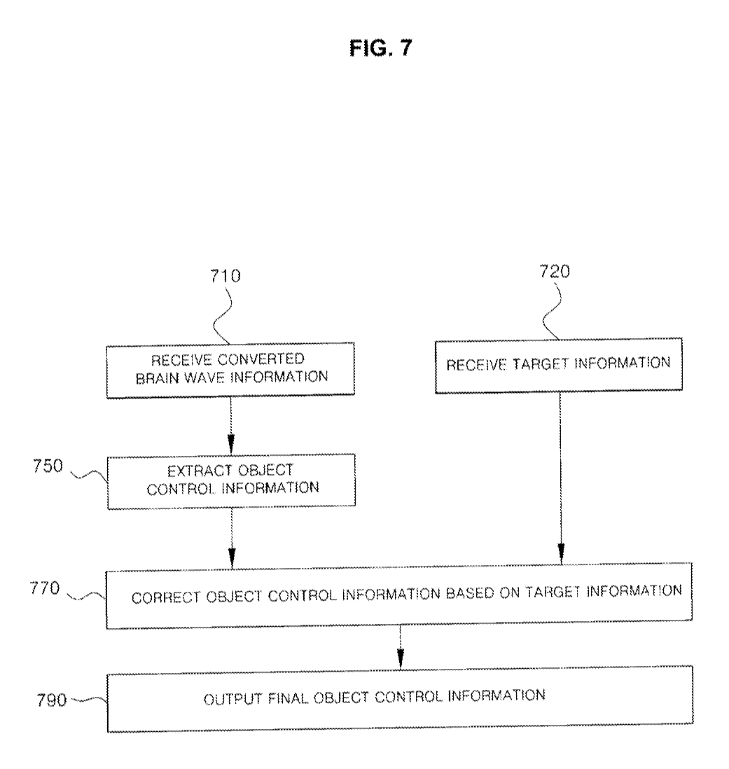

A brain-computer interface method in accordance with an exemplary embodiment of the present invention shown in FIG. 7 comprises a step 710 of receiving converted brain wave information, a step 750 of extracting object control information, a step 720 of receiving target information, a step 770 of correcting the object control information based on using the target information, and a step 790 of outputting final object control information.

In the step 710 of receiving the converted brain wave information, the converted brain wave information including object motion information is received.

In the step 750 of extracting the object control information, the object control information including object recognition information and object motion information is extracted from the converted brain wave information. The object control information is extracted from the converted brain wave information obtained by extracting motion information of an object (i.e., object motion information) that the subject wants to control from brain wave signals measured from the subject.

In the step 720 of receiving the target information, the target information including target location information of a target is received. The target means a final target, not a target candidate, and the received target information may be target information on at least one target. Moreover, the target information may include target recognition information.

In the step 770 of correcting the object control information based on the target information, the object control information is corrected using the target information.

In the step 790 of outputting the final object control information, the final object control information obtained by correcting the object control information based on the target information is output.

The target location information of the target information and the object motion information may be expressed as the distribution of probability values as shown in FIG. 12, not as explicit numerical values. In this case, the final object control information may be obtained by correcting the object control information based on volume distribution.

The final object control information may be continuously changed and determined based on the movement of the object, the change of the object control information extracted from the converted brain wave information, and the resulting change of the target information of target candidates.

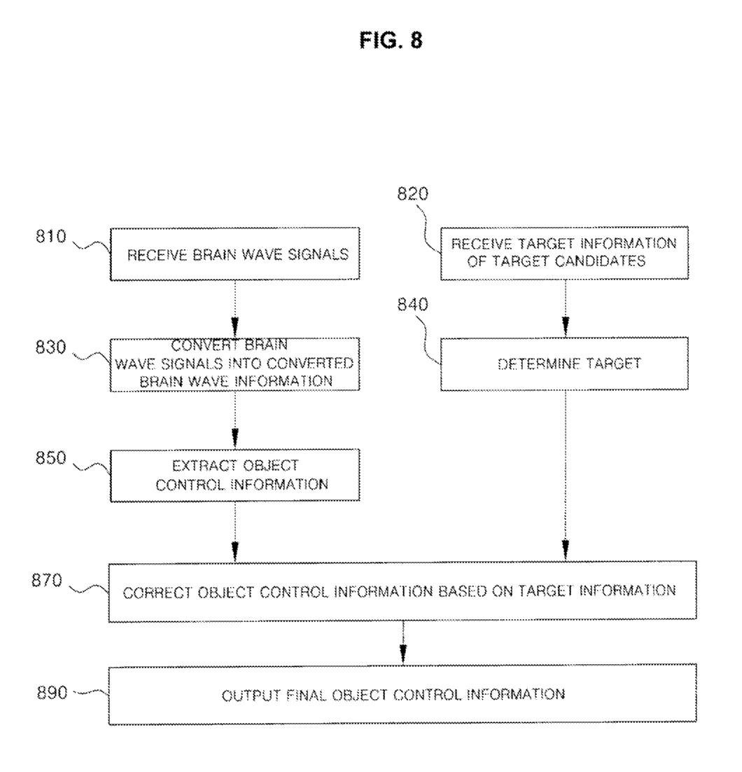

A brain-computer interface method in accordance with an exemplary embodiment of the present invention shown in FIG. 8 further comprises a step 810 of receiving brain wave signals, a step 830 of converting the brain wave signals into converted brain wave information, a step 820 of receiving target information of target candidates, and a step 840 of determining a target.

In the step 810 of receiving the brain wave signals, the brain wave signals such as EEG, MEG, etc. measured from the subject.

In the step 830 of converting the brain wave signals into the converted brain wave information, the received brain wave signals are converted into the converted brain wave information based on the object motion information, etc.

In the step 820 of receiving the target information of the target candidates, the target information including target location information of target candidates present around the subject or the object is received. Moreover, the target information may include target recognition information.

In the step 840 of determining the target, the target for the object to be controlled is determined from the target information on at least one target candidate. The determined target may be at least one target.

The step 830 of converting the brain wave signals into the converted brain wave information may comprise a signal processing process including a feature extraction process on the received brain wave or the brain wave signals subjected to preprocessing such as noise removal, etc. and a data classification process including a process of determining the object motion information based on the extracted features.

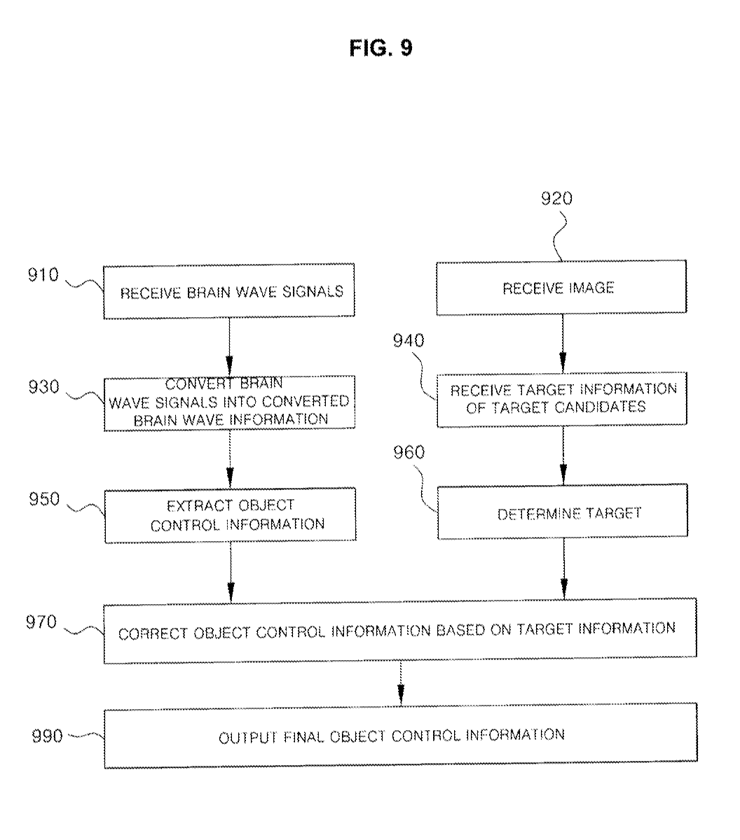

A brain-computer interface method in accordance with an exemplary embodiment of the present invention shown in FIG. 9 further comprises a step 920 of receiving an image and a step 940 of extracting target information of target candidates.

In the step 920 of receiving the image, the image of objects present around the subject or the object is received.

The received image is a surrounding image of the subject, from whom the brain wave signals are measured, and in particular a surrounding image in the direction of the subject's head or eyes may be suitable.

In the step 940 of extracting the target information of the target candidates, the target information including target location information of the target candidates is extracted from the received image by an image preprocessing process or an image processing technique of clustering the objects and based on information on the location and shape of the objects identified from the received image. Moreover, the target information may include target recognition information.

The received image may be a stereo image taken by a stereo camera and the target location information may be three-dimensional location information generated using depth information obtained from the stereo image.

As described above, according to the present invention, it is possible to provide a brain-computer interface using brain waves of a subject and to control an object.

Moreover, according to the present invention, it is possible to increase the accuracy of control of an object using target information in the brain-computer interface.

Furthermore, according to the present invention, it is possible to increase the accuracy of control of an object using image recognition of a target in the brain-computer interface.

In addition, according to the present invention, it is possible to increase the accuracy of determination of a target based on the object and the conditions of the object in the brain-computer interface.

While the invention has been particularly shown and described with reference to exemplary embodiments thereof, it will be understood by those of ordinary skill in the art that various changes in form and details may be made therein without departing from the spirit and scope of the invention as defined by the following claims.

What is claimed is:

1. A brain-computer interface device comprising:
a brain wave information processing unit which receives converted brain wave information including object motion information, extracts object control information including the object motion information from the converted brain wave information, and transmits the extracted object control information to a hybrid control unit; and
a hybrid control unit which receives target information including target location information of a target and outputs final object control information obtained by correcting the object control information including the object motion information based on the target information.

2. The brain-computer interface device of claim 1, wherein the object is any one of an artificial arm, a mouse cursor, a control means of an application program displayed on a display, a control means of an audio or video reproducing device, a wheelchair, and a vehicle.

3. The brain-computer interface device of claim 1, further comprising a brain wave signal conversion unit which receives brain wave signals from human, converts the received brain wave signals into converted brain wave information including object motion information, and transmits the converted brain wave information to the brain wave information processing unit.

4. The brain-computer interface device of claim 3, further comprising a brain wave signal preprocessing unit which receives the brain wave signals, removes noise signals from the brain wave signals, and transmits the resulting signals to the brain wave signal conversion unit.

5. The brain-computer interface device of claim 1, further comprising a target determination unit which receives target information including target location information on at least one target candidate, determines a target, and transmits the target information of the determined target to the hybrid control unit.

6. The brain-computer interface device of claim 5, further comprising an image recognition unit which receives an image, extracts at least one target candidate from the received image, sets target information including target location information of the target candidates, and transmits the target information to the target determination unit.

7. The brain-computer interface device of claim 6, wherein the received image is a stereo image taken by a stereo camera and the target location information is three-dimensional location information.

8. A brain-computer interface method comprising:
receiving converted brain wave information including object motion information;
extracting object control information including object motion information from the converted brain wave information;
receiving target information including target location information on a target; and
outputting final object control information obtained by correcting the object control information including the object motion information based on the target information.

9. The brain-computer interface method of claim 8, wherein the object is any one of an artificial arm, a mouse cursor, a control means of an application program displayed on a display, a control means of an audio or video reproducing device, a wheelchair, and a vehicle.

10. The brain-computer interface method of claim 8, further comprising, before receiving the converted brain wave information, receiving brain wave signals and converting the received brain wave signals into converted brain wave information including object motion information.

11. The brain-computer interface method of claim 10, further comprising, before converting the received brain wave signals into converted brain wave information, removing noise signals from the received brain wave signals.

12. The brain-computer interface method of claim 8, further comprising, before receiving the target information, receiving target information including target location information on at least one target candidate and determining a target.

13. The brain-computer interface method of claim 12, further comprising, before receiving the target information on at least one target candidate, receiving an image, extracting at least one target candidate from the received image, and setting target information including target location information of the target candidates based on the received image.

14. The brain-computer interface method of claim 13, wherein the received image is a stereo image taken by a stereo camera and the target location information is three-dimensional location information.

15. A computer-readable medium on which the brain-computer interface method of claim 8 is recorded in a program.
